SEQUENCE LISTING (2 sequences)

SEQ ID NO: 1 - protein, 10 aa, Artificial Sequence
  Synthetic
GGGGSGGGGS

SEQ ID NO: 2 - protein, 5 aa, Artificial sequence
  Synthetic
  REPEAT (1)..(5): may be repeated 1 or more times
GGGGS

(19) United States
(12) Patent Application Publication
(10) Pub. No.: US 2025/0236652 A1
(43) Pub. Date: Jul. 24, 2025

(54) SYSTEMS AND METHODS FOR IMMUNOGENIC CELL DEATH APPLICATIONS

(71) Applicant: Viska Bio Inc., Cambridge, MA (US)

(72) Inventor: Eric T. Fossel, Cambridge, MA (US)

(73) Assignee: Viska Bio, Inc., Cambridge, MA (US)

(21) Appl. No.: 18/848,622

(22) PCT Filed: Mar. 23, 2022
(86) PCT No.: PCT/US2022/021506

§ 371 Date: Sep. 19, 2024

(51) Int. Cl. C07K 14/47; C07K 16/00

(52) U.S. Cl. CPC C07K 14/4728; C07K 16/00; C07K 2319/33

(57) ABSTRACT

The present disclosure generally relates to systems and methods for treating and preventing diseases or conditions such as aging, atherosclerosis, obesity, viral disease, etc. These may be treated, according to some embodiments, by activation of an immune response within a subject. In some embodiments, for example, aging may be treated or prevented by removal of senescent cells by the immune system. In some embodiments, atherosclerosis may be treated or prevented by removal of plaque by the immune system. In some embodiments, obesity may be treated by removal of fat cells by the immune system. In some embodiments, viral diseases may be treated by removal of viruses by the immune system. Exemplary viral diseases include diseases caused by SARS-COV-2 and HIV, among others.

### TECHNICAL FIELD

Vaccines and methods of treatment for a variety of diseases are generally described. The present disclosure generally relates to systems and methods for treating and prevention of diseases or conditions such as aging, atherosclerosis, obesity, and viral diseases such as those caused by SARS-COV-2, HIV, influenza, SARS, and others.

### BACKGROUND

The immune system is designed to recognize and kill substances foreign to the body, including mutant cells, malignant cells, bacteria, viruses, etc. Both treatment and prevention of disease or conditions is important. Treatment and prevention of diseases or conditions such as aging, atherosclerosis, obesity, or viral disease takes many forms with vaccines being a primary prevention strategy in some cases, but with treatment often illusive. Accordingly, improvements are still needed.

### SUMMARY

Vaccines and methods of treatment for a variety of diseases or conditions are generally described. The present disclosure generally relates to systems and methods for treating and prevention of diseases or conditions such as aging, atherosclerosis, obesity, and viral diseases such as those caused by SARS-COV-2, HIV, influenza, SARS, and others. For example, it is the purpose of certain embodiments of the disclosure to treat and/or cure such diseases or conditions. Some embodiments are directed to vaccinating the body against the further recurrence or incidence of a disease or condition. As discussed in more detail herein, this may be accomplished in certain cases by using compositions containing moieties recognized by antigen presenting cells (e.g., dendritic cells) and portions of a virus acting as antigens; moieties recognized by antigen presenting cells (e.g., dendritic cells) and portions of plaque proteins and lipids acting as antigens; moieties recognized by antigen presenting cells (e.g., dendritic cells) and portions of fat cells acting as antigens; moieties recognized by antigen presenting cells (e.g., dendritic cells) and portions of virus acting as antigens; or other compositions such as those described herein. The composition may be taken up in some embodiments by antigen presenting cells and carried to lymph nodes, where the antigens may be processed and taught to T cells.

Several methods are disclosed herein of administering a subject with a composition for prevention or treatment of a particular condition or disease. It is to be understood that in each such aspect of the disclosure, the disclosure specifically includes, also, the composition for use in the treatment or prevention of that particular condition or disease, as well as use of the composition for the manufacture of a medicament for the treatment or prevention of that particular condition or disease.

In one aspect, the disclosure is directed towards a composition for treating or preventing a disease or condition. In some embodiments the composition comprises: a fusion protein comprising an eat-me signal and at least a portion of an antibody, wherein the at least a portion of the antibody is configured to bind to a cell, a virus, or an extracellular material associated with a disease or condition.

In another aspect, the disclosure is directed towards a method of treating or preventing a disease or condition. In some embodiments, the method comprises: administering to a subject a composition comprising a fusion protein that comprises an eat-me signal and an at least a portion of an antibody.

In yet another aspect, the disclosure is directed towards a composition for treating or preventing a disease or condition. In some embodiments, the composition comprises: a fusion protein comprising an eat-me signal and a fragment of a surface antigen of a cell or virus, wherein the composition is configured to trigger an immune response against the cell or the virus.

In still another aspect, the disclosure is directed towards a method of treating or preventing a disease or condition. In some embodiments, the method, comprises: administering to a subject a composition comprising a fusion protein that comprises an eat-me signal and a fragment of a surface antigen of a cell or virus.

In another aspect, the disclosure is directed towards a composition. In some embodiments, the composition comprises: an antibody that recognizes a cell, a virus, or an extracellular material, and an eat-me signal that is recognized by antigen presenting cells such as dendric cells.

In still another aspect, the disclosure is directed towards a composition. In some embodiments, the composition comprises: an antibody fragment that recognizes particular proteins of a cell, virus, or extracellular material and an eat-me signal that is recognized by antigen presenting cells such as dendric cells.

In yet another aspect, the disclosure is directed towards a method, comprising: administering, to a subject, a composition comprising an antibody that recognizes a cell, virus, or extracellular material and an eat-me signal.

In one aspect, the disclosure is directed towards a method, comprising: administering, to a subject, a composition comprising an antibody fragment that recognizes particular proteins of a cell or virus and an eat-me signal.

In another aspect, the disclosure is directed towards a composition comprising: an eat-me signal and one or more fragments of a surface antigen of a cell or virus.

In yet another aspect, the disclosure is directed towards a composition comprising: an eat-me signal and one or more DNA fragments of a cell or virus.

In still another aspect, the disclosure is directed towards a composition comprising: an eat-me signal and one or more lipid fragments of a cell or virus.

In another aspect, the disclosure is directed towards a composition comprising: an eat-me signal, a first component that is a DNA fragment, a lipid fragment, or a fragment of a surface antigen of a cell or virus, and a second component, different from the first component, that is also a DNA fragment, a lipid fragment, or a fragment of a surface antigen of a cell or virus.

Other advantages and novel features of the present disclosure will become apparent from the following detailed description of various non-limiting embodiments of the disclosure when considered in conjunction with the accompanying figures. In cases where the present specification and a document incorporated by reference include conflicting and/or inconsistent disclosure, the present specification shall control.

### DETAILED DESCRIPTION

The present disclosure generally relates to compositions and methods for treating and preventing diseases or conditions such as aging, atherosclerosis, obesity, viral disease, etc. These may be treated, according to some embodiments, by activation of an immune response within a subject. In some embodiments, for example, aging may be treated or prevented by removal of senescent cells by the immune system. In some embodiments, atherosclerosis may be treated or prevented by removal of plaque by the immune system. In some embodiments, obesity may be treated by removal of fat cells by the immune system. In some embodiments, viral diseases may be treated by removal of viruses by the immune system. Exemplary viral diseases include diseases caused by SARS-COV-2 and HIV, among others.

The compositions and methods of the present disclosure are, in a number of aspects, directed towards the treatment of diseases or conditions of a subject (e.g., a person or a non-human animal) using the subject's immune system. For example, the subject's immune system may be employed, in some embodiments, to phagocytose cells, virses, or extracellular materials that cause the disease or condition. In some embodiments, compositions such as those described herein are configured to target cells, viruses, or extracellular materials for phagocytosis by directly binding to the cells, viruses, or extracellular materials. Targeting cells, viruses, or extracellular materials for phagocytosis may treat the disease or condition. Targeting cells, viruses, or extracellular materials for phagocytosis may also train the subject's immune system to recognize and target the cells, viruses, or extracellular materials on its own, so that even in the eventual absence of the composition, the subject's immune system continues to phagocytose the cells, viruses, or extracellular materials. In another aspect, a composition is provided for the purpose of training an immune response against disease- or condition-causing cells, bacteria, or extracellular materials. The composition may comprise fragments (e.g., surface antigen fragments) of the cells, bacteria, or extracellular materials, and may be configured to be phagocytosed by immune cells. Such compositions may be used, in some embodiments, to train an immune response against the cells, bacteria, or extracellular materials, thereby preventing or treating the disease or condition caused by the cells, bacteria, or extracellular materials.

In some environments, a composition comprises an “eat-me” signal. An eat-me signal, as is known to those of ordinary skill in the art, may refer to a signaling moiety (e.g., a protein or a polypeptide) that helps a phagocyte recognize extracellular cargo (for example, a cell, a virus, an extracellular material, or a fragment of a cell, a virus, or an extracellular material) for phagocytosis. Phagocytosis may refer to a process by which a cell engulfs and breaks down extracellular cargo. Without wishing to be bound by theory, phagocytosis may be used by the immune system as a mechanism for removing pathogens, dead cells, or other cell debris. Ordinarily, phagocytosis of living cells of a subject would be relatively unlikely. However, in the context of the present disclosure it has been inventively recognized that phagocytosis may be adapted for the treatment or prevention of diseases or conditions, including diseases or conditions caused by living cells of a subject.

The eat-me signal may be configured to induce a phagocyte to phagocytose a cell, a virus, or an extracellular material connected to the eat-me signal. For example, the eat-me signal may be configured to induce an antigen presenting cell to phagocytose the virus, the cell, or the extracellular material.

Any of a variety of eat-me signals may be used. For example, the eat-me signal may comprise a biopolymer. In some embodiments, the biopolymer comprises a polypeptide (e.g., a protein). For example, the eat-me signal may be a protein (e.g., calreticulin; an annexin such as annexin A1; a pentraxin such as pentraxin-3; or a histone) or a fragment thereof. In some embodiments, the biopolymer is a nucleic acid (e.g., DNA). In some embodiments, the eat-me signal is a small-molecule (e.g., a phospholipid, such as a physophatidylserine; an oxidized phospholipid; or a sugar residue, such as galactose). In the context of the present disclosure, it has been recognized that the use of a polypeptide eat-me signal (e.g., calreticulin) may be advantageous for the treatment or prevention of diseases or conditions. In some embodiments, a polypeptide eat-me signal forms a portion of a fusion protein, which may have a number of unexpected advantages, described in greater detail below.

Herein, it has been inventively recognized that in certain embodiments, cells, viruses, or extracellular materials that cause any of a variety of diseases or conditions may be targeted for early phagocytosis by binding eat-me signals to the cells, viruses, or extracellular materials for the purpose of labeling them for phagocytosis. In principle, labeling of a cell, virus, or extracellular material with eat-me signals can be achieved using any of a variety of known antibodies that recognize the cell, virus, or extracellular material, as discussed in greater detail below.

In some embodiments, a composition comprising an eat-me signal is suitable for treating a disease. Such a composition may be administered to a subject (e.g., a person) with the disease. In some embodiments, the composition will produce an immune response against the cell, virus, or extracellular material targeted by the antibody. In some embodiments, the immune response will treat the disease or condition by attacking the targeted cell, virus, or extracellular material, limiting the symptoms of the disease or condition. A subject treated using the composition may be at least partially immunized against the disease or condition.

In a first aspect, a composition comprising an eat-me signal and an antibody may be used to treat diseases or conditions by binding to cells, viruses, or extracellular material and triggering phagocytosis. Such a composition may be used to treat diseases, by triggering an immune response against cells, viruses, or extracellular materials causing those diseases. The following paragraphs provide specific examples.

In some embodiments, a composition (e.g., a composition comprising an eat-me signal) is configured to be connected to an extracellular material that causes atherosclerosis. For example, the eat-me signal of the composition may be configured to be connected to plaque. Without wishing to be bound by theory, plaque is generally a multicomponent material that may comprise cholesterol, plaque cells such as white blood cells, calcium, and other fatty substances. The plaque may form in or around the walls of blood vessels (e.g., arteries), and may thus cause atherosclerosis.

Herein, it has been inventively recognized that through the use of compositions comprising eat-me signals, plaque may be targeted for phagocytosis. Phagocytosis of the plaque may reduce the quantity of plaque, thereby treating the atherosclerosis. Furthermore, in some embodiments, the immune system may learn to recognize the plaque and target it for phagocytosis, even in the eventual absence of the composition. Thus, the composition may act as a vaccine against atherosclerosis, in some embodiments.

According to certain embodiments, a composition useful for treating or preventing atherosclerosis comprises at least a portion of an antibody (e.g., an entire antibody or an antibody fragment) that recognizes plaque. For example, the composition may comprise at least a portion of plaque-reactive antibody A12, ASA6, or any of a variety of plaque-recognizing antibodies known to those of ordinary skill in the art.

The at least a portion of the antibody (e.g., the entire antibody or the antibody fragment) and the eat-me signal may be connected to one another, as discussed in greater detail below. In some embodiments, the eat-me signal and the antibody form a fusion protein. The fusion protein may be particularly advantageous for treating or preventing arteriosclerosis. In some embodiments, for example, the fusion protein may bind to plaque with a higher affinity than would be expected from an identical eat-me signal connected to an identical antibody or antibody portion by a linker. In some embodiments, the fusion protein also has a longer lifetime in the subject, which may also result in an improved performance of the composition.

The antibody may be chosen to bind to any of a variety of appropriate extracellular materials within the plaque. For example, the antibody may be chosen to bind to cholesterol, plaque cells such as white blood cells, calcium, and or other fatty substances in the plaque.

In some embodiments, the composition may be used to treat or prevent the condition of aging. For example, the eat-me signal of the composition may be configured to be connected to a senescent cell. Without wishing to be bound by theory, senescent cells may be incapable of cell division, and/or may be associated with diseases of aging in certain embodiments.

Herein, it has been inventively recognized that through the use of compositions comprising eat-me signals, senescent cells may be targeted for phagocytosis. The phagocytosis of senescent cells may reduce the quantity or proportion of senescent cells within the body. The reduction in the quantity or proportion of senescent cells may, in turn, retard or reverse the aging process. Furthermore, in some embodiments, the immune system may learn to recognize senescent cells and target them for phagocytosis, even in the eventual absence of the composition. Thus, the composition may act as a vaccine against aging and related diseases, in some embodiments.

According to certain embodiments, a composition useful for treating or preventing aging comprises at least a portion of an antibody (e.g., an entire antibody or an antibody fragment) that recognizes senescent cells. For example, the composition may comprise at least a portion of canakinumab, an anti-DPP4 antibody, an anti-CD9 antibody, or another of a variety of appropriate antibodies that recognize senescent cells and are known to those of ordinary skill in the art.

The at least a portion of the antibody (e.g., the entire antibody or the antibody fragment) and the eat-me signal may be connected to one another, as discussed in greater detail below. In some embodiments, the eat-me signal and the antibody form a fusion protein. The fusion protein may be particularly advantageous for treating or preventing aging. In some embodiments, for example, the fusion protein may bind to senescent cells with a higher affinity than would be expected from an identical eat-me signal connected to an identical antibody or antibody portion by a linker. In some embodiments, the fusion protein also has a longer lifetime in the subject, which may also result in an improved performance of the composition.

In some embodiments, the composition may be used to treat or prevent the disease of obesity. For example, the eat-me signal of the composition may be configured to be connected to a fat cell (also known as an adipocyte). An overabundance of fat cells and/or an overabundance of over-sized fat cells may be associated with obesity, as well as a number of related conditions, such as insulin resistance/diabetes.

Herein, it has been inventively recognized that through the use of compositions comprising eat-me signals, fat cells may be targeted for phagocytosis. Phagocytosis of the fat cells may reduce the number of fat cells in the body, thereby treating obesity. Furthermore, in some embodiments, the immune system may learn to recognize fat cells and target them for phagocytosis, even in the eventual absence of the composition. Thus, the composition may act as a vaccine against obesity and related diseases, in some embodiments.

According to certain embodiments, a composition useful for treating or preventing obesity comprises at least a portion of an antibody (e.g., an entire antibody or an antibody fragment) that recognizes fat cells. For example, the composition may comprise at least a portion of an adipocyte-specific IgG antibody, an adipocyte-type fatty acid-binding protein (A-FABP), an anti-ASC-1 antibody, an anti-PAT2 antibody, an anti-P2RX5 antibody, or another of a variety of appropriate antibodies that recognize fat cells and are known to those of ordinary skill in the art.

The at least a portion of the antibody (e.g., the entire antibody or the antibody fragment) and the eat-me signal may be connected to one another, as discussed in greater detail below. In some embodiments, the eat-me signal and the antibody form a fusion protein. The fusion protein may be particularly advantageous for treating or preventing obesity. In some embodiments, for example, the fusion protein may bind to fat cells with a higher affinity than would be expected from an identical eat-me signal connected to an identical antibody or antibody portion by a linker. In some embodiments, the fusion protein also has a longer lifetime in the subject, which may also result in an improved performance of the composition.

In some embodiments, the composition may be used to treat or prevent the viral diseases. For example, the eat-me signal of the composition may be configured to be connected to a virus. Exemplary viruses include coronaviruses (e.g., SARS-COV-2), rhinoviruses, influenza viruses, HIV, or any of a variety of other virus types. The compositions and methods described herein may have a number of advantages for preventing diseases caused by these viruses, as discussed below.

Herein, it has been inventively recognized that through the use of compositions comprising eat-me signals, viruses may be targeted for phagocytosis. The phagocytosis of viruses may reduce the quantity or proportion of viruses within the body. The reduction in the number of viruses may in turn reduce the intensity of symptoms caused by the virus, thereby treating the virus. Furthermore, in some embodiments, the immune system may learn to recognize the virus and target it for phagocytosis, even in the eventual absence of the composition. Thus, the composition may act as a vaccine against the virus and related diseases, in some embodiments.

According to certain embodiments, a composition useful for treating or preventing viral disease comprises at least a portion of an antibody (e.g., an entire antibody or an antibody fragment) that recognizes a virus. For example, the composition may comprise at least a portion of an antibody that recognizes SARS COV-2, such as casirivimab, imdevimab, etesevimab, bamlanivimab, or sotrovimab; an antibody that recognizes HIV, such as ibalizumab, 10-1074, or 3BNC117; or any of a variety of other virus recognizing antibodies known to those of ordinary skill in the art.

The at least a portion of the antibody (e.g., the entire antibody or the antibody fragment) and the eat-me signal may be connected to one another, as discussed in greater detail below. In some embodiments, the eat-me signal and the antibody form a fusion protein. The fusion protein may be particularly advantageous for treating or preventing viral disease. In some embodiments, for example, the fusion protein may bind to viruses with a higher affinity than would be expected from an identical eat-me signal connected to an identical antibody or antibody portion by a linker. In some embodiments, the fusion protein also has a longer lifetime in the subject, which may also result in an improved performance of the composition.

In another aspect, a composition comprises an eat-me signal and a fragment from a disease-causing virus, cell, or extracellular material (e.g., a senescent cell, a fat cell, plaque, or a virus, etc.), triggering phagocytosis of the composition. Any appropriate fragment may be used. For example, the fragment may comprise a biopolymer such as a polypeptide (e.g., a protein or a fragment thereof) or a polynucleotide (e.g., DNA, RNA). In some embodiments, the fragment is a small molecule (e.g., a component of plaque). In some embodiments, the fragment is a fragment of a surface antigen of a cell or virus. Such a composition may be used to train the immune system to target the disease-causing virus, cell, or extracellular material, by triggering an immune response against cells, viruses, or extracellular materials including the fragment bound to the eat-me signal of the composition. The paragraphs below provide a number of specific examples.

In some embodiments, a composition (e.g., a composition comprising an eat-me signal) is configured to be connected to a fragment of an extracellular material that causes atherosclerosis. For example, the eat-me signal of the composition may be configured to be connected to a fragment of plaque. In some embodiments, the fragment is a fragment of a surface antigen of a cell or virus found in plaque.

Herein, it has been inventively recognized that through the use of compositions comprising eat-me signals and fragments of plaque, phagocytes may be trained to target plaque for phagocytosis. Phagocytosis of the plaque may reduce the quantity of plaque, thereby treating or preventing atherosclerosis. Thus, the composition may act as a vaccine against atherosclerosis, in some embodiments.

Suitable fragments of plaque that may be comprised by the composition include aldehyde dehydrogenase 4 family member A1 (ALDH4A1) or any of a variety of other fragments of plaque known to those of ordinary skill in the art

As discussed elsewhere herein, in some embodiments, the composition may comprise a fusion protein. The fusion protein may be particularly advantageous for preventing arteriosclerosis. In some embodiments, the fusion protein also has a longer lifetime in the subject, which may also result in an improved performance of the composition.

In some embodiments, a composition (e.g., a composition comprising an eat-me signal) is configured to be connected to a fragment of a senescent cell. For example, the fragment may be a fragment of a surface antigen of the senescent cell. Herein, it has been inventively recognized that through the use of compositions comprising eat-me signals and fragments of senescent cells, phagocytes may be trained to target senescent cells for phagocytosis. Phagocytosis of the senescent cells may reduce the quantity of senescent cells, thereby treating or preventing aging. Thus, the composition may act as a vaccine against aging, in some embodiments.

Suitable fragments of senescent cells that may be comprised by the composition include CD9 receptors, DPP4, or any of a variety of other fragments of senescent cells known to those of ordinary skill in the art.

As discussed elsewhere herein, in some embodiments, the composition may comprise a fusion protein. The fusion protein may be particularly advantageous for preventing aging. In some embodiments, the fusion protein also has a longer lifetime in the subject, which may also result in an improved performance of the composition.

In some embodiments, a composition (e.g., a composition comprising an eat-me signal) is configured to be connected to a fragment of a fat cell. For example, the fragment may be a fragment of a surface antigen of the fat cell. Herein, it has been inventively recognized that through the use of compositions comprising eat-me signals and fragments of fat cells, phagocytes may be trained to target fat cells for phagocytosis. Phagocytosis of the fat cells may reduce the quantity of fat cells (and/or the quantity of oversized fat cells), thereby treating or preventing obesity. Thus, the composition may act as a vaccine against obesity and/or related diseases, such as diabetes, in some embodiments.

Suitable fragments of fat cells that may be comprised by the composition include. an adipocyte-type fatty acid, ASC-1, PAT2, P2RX5, or any of a variety of other fragments of fat cells known to those of ordinary skill in the art.

As discussed elsewhere herein, in some embodiments, the composition may comprise a fusion protein. The fusion protein may be particularly advantageous for preventing obesity. In some embodiments, the fusion protein also has a longer lifetime in the subject, which may also result in an improved performance of the composition.

In some embodiments, a composition (e.g., a composition comprising an eat-me signal) is configured to be connected to a fragment of a virus. For example, the fragment may be a fragment of a surface antigen of the virus. Herein, it has been inventively recognized that through the use of compositions comprising eat-me signals and fragments of viruses, phagocytes may be trained to target viruses for phagocytosis. Phagocytosis of the viruses may reduce the quantity of viruses, thereby treating or preventing viral disease. Thus, the composition may act as a vaccine against viral disease, in some embodiments.

Suitable fragments of viruses that may be comprised by the composition include the SARS-COV-2 spike protein, CD4 receptors of the HIV virus, or any of a variety of other fragments of viruses known to those of ordinary skill.

As discussed elsewhere herein, in some embodiments, the composition may comprise a fusion protein. The fusion protein may be particularly advantageous for preventing viral disease. In some embodiments, the fusion protein also has a longer lifetime in the subject, which may also result in an improved performance of the composition.

The eat-me signal may be connected to another portion of the composition (e.g., an antibody, an antibody portion, or a fragment of a disease-causing cell, virus, or extracellular material, etc.) covalently. In some embodiments, the eat-me signal and the other portion of the composition (e.g., an antibody, an antibody portion, or a fragment of a disease-causing cell, virus, or extracellular material) are directly connected, such that they form a single polypeptide chain. For example, the composition may be expressed as a single fusion protein containing both an eat-me signal (e.g., calreticulin) and another portion of the composition (e.g., an antibody, an antibody portion, or a fragment of a disease-causing cell, virus, or extracellular material). In other embodiments, however, other methods may be used to connect the eat-me signal to the composition, for example, directly bound to each other, or bound via one or more cross-linking agents. Non-limiting examples include glutaraldehyde, NHS-esters (N-hydroxysuccinimide) (e.g., dithiobis(succinimidylpropionate), dithiobis(sulfosuccinimidylpropionate), etc.), PEG groups, imidoesters (e.g., dimethyl adipimidate, dimethyl suberimidate, dimethyl pimelimidate, etc.), maleimides, pyridyls, carbodiimide, isocyanate, or the like. The antibody and the enzyme may be coupled through any suitable system, e.g., amine-to-amine, sulfhydryl-to-sulfhydryl, amine-to-sulfhydryl, carboxyl-to-amine, sulfhydryl-to-carbohydrate, hydroxyl-to-sulfhydryl, or the like. Those of ordinary skill in the art will be familiar with methods of cross-linking or conjugating proteins to each other.

A “fusion protein” generally refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A fusion protein may comprise different domains, for example, an eat-me signal and an antibody; an eat-me signal and an antibody fragment; a fragment of a disease-causing cell; a fragment of a virus; or a fragment of an extracellular protein. In some embodiments, the eat-me signal is fused at the N-terminus of the antibody, antibody fragment, fragment of a disease-causing cell, fragment of a virus, or fragment of an extracellular protein. In some embodiments, the eat-me signal is fused at the C-terminus of the antibody, antibody fragment, fragment of a disease-causing cell, fragment of a virus, or fragment of an extracellular protein.

In some embodiments, the eat-me signal and the antibody, antibody fragment, or fragments of a surface antigen of a cell or virus are separated from the antibody or antibody fragment by a linker region. The linker region may be configured to separate the antibody from the enzyme spatially. In some embodiments, the linker region may have an amino acid sequence that comprises repeated subsequences. For example, in some embodiments the linker region has an amino acid sequence that comprises (GGGGS)ₙ (SEQ ID NO 6), where is an integer greater than or equal to 1 (e.g., 1, 2, 3, 4, 5, 6, 8, 10, or more). In some embodiments, the linker region is GGGGSGGGGS (SEQ ID NO 1). In other embodiments, the composition does not comprise a linker region.

Further provided herein are eat-me signal variants and fusion proteins comprising such eat-me signal variants. In some embodiments, the fusion protein described herein comprises a modification. When the fusion protein is referred to herein, it encompasses all its variants and derivatives. Polypeptides comprising modifications have additional features other than amino acid contents. As used herein, a “modification” or “derivative” of a protein or polypeptide (e.g., the fusion protein described herein) produces a modified or derivatized polypeptide, which is a form of a given peptide that is chemically modified relative to the reference peptide, the modification including, but not limited to, oligomerization or polymerization, modifications of amino acid residues or peptide backbone, cross-linking, cyclization, conjugation, PEGylation, glycosylation, acetylation, phosphorylation, acylation, carboxylation, lipidation, thioglycolic acid amidation, alkylation, methylation, polyglycylation, glycosylation, polysialylation, adenylylation, PEGylation, fusion to additional heterologous amino acid sequences, or other modifications that substantially alter the stability, solubility, or other properties of the peptide while substantially retaining the activity of the polypeptides described herein. It is to be understood that the fusion protein comprising such modifications, are cross-linked, cyclized, conjugated, acylated, carboxylated, lipidated, acetylated, thioglycolic acid amidated, alkylated, methylated, polyglycylated, glycosylated, polysialylated, phosphorylated, adenylylated, PEGylated, or combination thereof. In some embodiments, the modified fusion protein of the present disclosure may contain non-amino acid elements, such as polyethylene glycols, lipids, poly- or mono-saccharide, and phosphates. The fusion protein of the present disclosure, may comprise the modifications disclosed herein at the C-terminus (e.g., C-terminal amidation), N-terminus (e.g., N-terminal acetylation). Terminal modifications are useful, and are well known, to reduce susceptibility to proteinase digestion, and therefore serve to prolong half-life of the polypeptides in solutions, particularly biological fluids where proteases may be present. In some embodiments, the fusion proteins described herein are further modified within the sequence, such as, modification by terminal-NH₂ acylation, e.g., acetylation, or thioglycolic acid amidation, by terminal-carboxylamidation, e.g., with ammonia, methylamine, and the like terminal modifications.

Terminal modifications are useful, to reduce susceptibility by proteinase digestion, and therefore can serve to prolong half-life of the polypeptides in solution, particularly in biological fluids where proteases may be present. Amino terminus modifications include methylation (e.g., —NHCH₃ or —N(CH₃)₂), acetylation (e.g., with acetic acid or a halogenated derivative thereof such as a-chloroacetic acid, a-bromoacetic acid, or a-iodoacetic acid), adding a benzyloxycarbonyl (Cbz) group, or blocking the amino terminus with any blocking group containing a carboxylate functionality defined by RCOO— or sulfonyl functionality defined by R—SO₂—, where R is selected from the group consisting of alkyl, aryl, heteroaryl, alkyl aryl, and the like, and similar groups. One can also incorporate a desamino acid at the N-terminus (so that there is no N-terminal amino group) to decrease susceptibility to proteases or to restrict the conformation of the polypeptide. In certain embodiments, the N-terminus is acetylated with acetic acid or acetic anhydride.

Carboxy terminus modifications include replacing the free acid with a carboxamide group or forming a cyclic lactam at the carboxy terminus to introduce structural constraints. One can also cyclize the peptides described herein, or incorporate a desamino or descarboxy residue at the termini of the peptide, so that there is no terminal amino or carboxyl group, to decrease susceptibility to proteases or to restrict the conformation of the peptide. Methods of circular peptide synthesis are known in the art. C-terminal functional groups of the peptides described herein include amide, amide lower alkyl, amide di(lower alkyl), lower alkoxy, hydroxy, and carboxy, and the lower ester derivatives thereof, and the pharmaceutically acceptable salts thereof.

In some embodiments, the fusion proteins described herein are phosphorylated. One can also readily modify peptides by phosphorylation, and other methods. In some embodiments, one can also replace the naturally occurring side chains of the genetically encoded amino acids (or the stereoisomeric D amino acids) with other side chains, for instance with groups such as alkyl, lower (C₁₋₆) alkyl, cyclic 4-, 5-, 6-, to 7-membered alkyl, amide, amide lower alkyl amide di(lower alkyl), lower alkoxy, hydroxy, carboxy and the lower ester derivatives thereof, and with 4-, 5-, 6-, to 7-membered heterocycles. For example, proline analogues in which the ring size of the proline residue is changed from 5 members to 4, 6, or 7 members can be employed.

Cyclic groups can be saturated or unsaturated, and if unsaturated, can be aromatic or non-aromatic. Heterocyclic groups preferably contain one or more nitrogen, oxygen, and/or sulfur heteroatoms. Examples of such groups include the furazanyl, furyl, imidazolidinyl, imidazolyl, imidazolinyl, isothiazolyl, isoxazolyl, morpholinyl (e.g. morpholino), oxazolyl, piperazinyl (e.g., 1-piperazinyl), piperidyl (e.g., 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g., 1-pyrrolidinyl), pyrrolinyl, pyrrolyl, thiadiazolyl, thiazolyl, thienyl, thiomorpholinyl (e.g., thiomorpholino), and triazolyl groups. These heterocyclic groups can be substituted or unsubstituted. Where a group is substituted, the substituent can be alkyl, alkoxy, halogen, oxygen, or substituted or unsubstituted phenyl.

In some embodiments, the fusion proteins described herein may be attached to one or more polymer moieties. In some embodiments, these polymers are covalently attached to the fusion proteins of the disclosure. In some embodiments, for therapeutic use of the end product preparation, the polymer is pharmaceutically acceptable. One skilled in the art will be able to select the desired polymer based on such considerations as whether the polymer-peptide conjugate will be used therapeutically, and if so, the desired dosage, circulation time, resistance to proteolysis, and other considerations.

All combinations of the different modifications and derivatizations are envisioned for the fusion protein described herein.

Other aspects of the present disclosure provide methods of producing the fusion protein. The fusion protein may be produced by expression form recombinant nucleic acids in appropriate cells (e.g., bacterial cell or eukaryotic cells) and isolated. To produce the fusion protein, nucleic acids encoding the fusion protein may be introduced to a cell (e.g., a bacterial cell or a eukaryotic cell such as a yeast cell or an insect cell. The cells may be cultured under conditions that allow the fusion protein to express from the nucleic acids encoding the fusion protein. Fusion proteins comprising a signal peptide can be secreted, e.g., into the culturing media and can subsequently be recovered. The fusion protein may be isolated using any methods of purifying a protein known in the art.

The nucleic acids encoding the fusion protein described herein may be obtained, and the nucleotide sequence of the nucleic acids determined, by any method known in the art. One skilled in the art is able to identify the nucleotide sequence encoding the fusion protein from the amino acid sequence of the fusion protein. The nucleic acids encoding the fusion protein of the present disclosure, may be DNA or RNA, double-stranded or single stranded. In some embodiments, the nucleotide sequence encoding the fusion protein may be codon optimized to adapt to different expression systems (e.g., for mammalian expression).

In some embodiments, the nucleic acid is comprised within a vector, such as an expression vector. In some embodiments, the vector comprises a promoter operably linked to the nucleic acid.

A variety of promoters can be used for expression of the fusion proteins described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter.

Regulatable promoters can also be used. Such regulatable promoters include those using the lac repressor from E. coli as a transcription modulator to regulate transcription from lac operator-bearing mammalian cell promoters, those using the tetracycline repressor (tetR), etc. Other systems include FK506 dimer, VP16 or p65 using astradiol, RU486, diphenol murislerone, or rapamycin.

Regulatable promoters that include a repressor with the operon can be used. In one embodiment, the lac repressor from Escherichia coli can function as a transcriptional modulator to regulate transcription from lac operator-bearing mammalian cell promoters. In one embodiment, a tetracycline inducible switch is used.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

An expression vector comprising the nucleic acid can be transferred to a host cell by conventional techniques (e.g., electroporation, liposomal transfection, and calcium phosphate precipitation) and the transfected cells are then cultured by conventional techniques to produce the fusion proteins described herein. In some embodiments, the expression of the fusion proteins described herein is regulated by a constitutive, an inducible or a tissue-specific promoter.

The host cells used to express the fusion proteins described herein may include bacterial cells such as Escherichia coli, eukaryotic cells, or he like. In one embodiment, mammalian cells, such as Chinese hamster ovary cells (CHO) can be used.

A variety of host-expression vector systems may be utilized to express the fusion proteins described herein. Such host-expression systems represent vehicles by which the coding sequences of the isolated fusion proteins described herein may be produced and subsequently purified, but also represent cells which may, when transformed or transfected with the appropriate nucleotide coding sequences, express the fusion proteins described herein in situ. These include, but are not limited to, microorganisms such as bacteria (e.g., E. coli and B. subtilis) transformed with recombinant bacteriophage DNA, plasmid DNA or cosmid DNA expression vectors containing coding sequences for the fusion proteins described herein; yeast (e.g., Saccharomyces pichia) transformed with recombinant yeast expression vectors containing sequences encoding the fusion proteins described herein; insect cell systems infected with recombinant virus expression vectors (e.g., baclovirus) containing the sequences encoding the fusion proteins described herein; plant cell systems infected with recombinant virus expression vectors (e.g., cauliflower mosaic virus (CaMV) and tobacco mosaic virus (TMV) or transformed with recombinant plasmid expression vectors (e.g., Ti plasmid) containing sequences encoding the fusion proteins described herein; or mammalian cell systems (e.g., COS, CHO, BHK, 293, 293T, 3T3 cells, lymphotic cells, Per C.6 cells harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the fusion proteins being expressed. For example, when a large quantity of such a protein is to be produced, for the generation of pharmaceutical compositions of fusion proteins described herein, vectors which direct the expression of high levels of fusion protein products that are readily purified may be desirable. Such vectors include, but are not limited, to the E. coli expression vector pUR278, in which the coding sequence may be ligated individually into the vector in frame with the lac Z coding region so that a fusion protein is produced; pIN vectors, and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In mammalian host cells, a number of viral-based expression systems may be utilized. In cases where an adenovirus is used as an expression vector, the coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the immunoglobulin molecule in infected hosts. Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc.

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Purification and modification of recombinant proteins is well known in the art such that the design of the polyprotein precursor could include a number of embodiments readily appreciated by a skilled worker. Any known proteases or peptidases known in the art can be used for the described modification of the precursor molecule, e.g., thrombin or factor Xa, enterokinase, furin, and AcTEV, and the Foot and Mouth Disease Virus Protease C3, etc.

Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, HeLa, COS, MDCK, 293, 293T, 3T3, WI38, BT483, Hs578T, HTB2, BT20 and T47D, CRL7030 and Hs578Bst.

For long-term, high-yield production of recombinant proteins, stable expression is preferred. For example, cell lines which stably express fusion proteins described herein may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the fusion proteins described herein. Such engineered cell lines may be particularly useful in screening and evaluation of fusion proteins that interact directly or indirectly with the fusion proteins described herein.

The expression levels of the fusion described herein can be increased by vector amplification. When a marker in the vector system expressing a fusion protein described herein is amplifiable, increase in the level of inhibitor present in culture of host cell will increase the number of copies of the marker gene. Since the amplified region is associated with the nucleotide sequence of a fusion protein described herein or a fusion protein described herein, production of the fusion protein will also increase.

Once a fusion described herein has been recombinantly expressed, it may be purified by any method known in the art for purification of polypeptides, polyproteins or antibodies (e.g., analogous to antibody purification schemes based on antigen selectivity) for example, by chromatography (e.g., ion exchange, affinity, particularly by affinity for the specific antigen (optionally after Protein A selection where the polypeptide comprises an Fc domain (or portion thereof)), and sizing column chromatography), centrifugation, differential solubility, or by any other standard technique for the purification of polypeptides or antibodies.

In some embodiments, fusion proteins may advantageously resist degradation, relative to other compositions comprising connected antibodies and eat-me signals. Fusion proteins may also result in unexpectedly high performance of the molecule. For example. For example, the eat-me signal, the antibody, the antibody fragment, the fragment of the disease-causing cell, the fragment of the virus, or the fragment of the extracellular protein may have an unexpectedly high activity when comprised by a fusion protein of a composition, relative to the activity of compositions where the eat-me signal is cross-linked with the antibody, the antibody fragment, the fragment of the disease-causing cell, the fragment of the virus, or the fragment of the extracellular protein. This may advantageously improve the potency of compositions comprising fusion proteins.

The treatments disclosed herein may be given to any subject, for example, a human, or a non-human animal, such as a dog, a cat, a horse, a rabbit, a cow, a pig, a sheep, a goat, a rat (e.g., Rattus Norvegicus), a mouse (e.g., Mus musculus), a guinea pig, a non-human primate (e.g., a monkey, a chimpanzee, a baboon, an ape, a gorilla, etc.), or the like.

However, the eat-me signal may be connected to another portion of the composition (e.g., an antibody, an antibody portion, or a fragment of a disease-causing cell, virus, or extracellular material) via cross-linking. The portion and the eat-me signal may be crosslinked using a crosslinking agent. The cross-link may comprise a synthetic cross-linker, or may comprise a direct coupling between amino-acid side-chains of the antibody and the eat-me signal. In some embodiments, the antibody and the eat-me signal are cross-linked using one or more cross-linking agents. Non-limiting examples of cross-linking agents include glutaraldehyde, NHS-esters (N-hydroxysuccinimide) (e.g., dithiobis(succinimidylpropionate), dithiobis(sulfosuccinimidylpropionate), etc.), PEG groups, imidoesters (e.g., dimethyl adipimidate, dimethyl suberimidate, dimethyl pimelimidate, etc.), maleimides, pyridyls, carbodiimide, isocyanate, or the like.

The antibody and the eat-me signal may be coupled through any suitable coupling system, e.g., amine-to-amine, sulfhydryl-to-sulfhydryl, amine-to-sulfhydryl, carboxyl-to-amine, sulfhydryl-to-carbohydrate, hydroxyl-to-sulfhydryl, or the like. Those of ordinary skill in the art will be familiar with methods of cross-linking or conjugating proteins to each other.

The compositions described herein may be administered to the subject (e.g., a human) using any of a variety of suitable techniques, as discussed in greater detail below. The composition may be administered to the subject in any suitable dose. For example, the composition may be administered in a dosage of greater than or equal to 10 mg, greater than or equal to 15 mg, greater than or equal to 20 mg, greater than or equal to 25 mg, greater than or equal to 30 mg, greater than or equal to 40 mg, greater than or equal to 50 mg, greater than or equal to 60 mg, greater than or equal to 70 mg, greater than or equal to 80 mg, greater than or equal to 90 mg, greater than or equal to 100 mg, greater than or equal to 200 mg, greater than or equal to 300 mg, greater than or equal to 500 mg, greater than or equal to 1000 mg, greater than or equal to 1500 mg, greater than or equal to 2000 mg, greater than or equal to 2500 mg, greater than or equal to 3000 mg, greater than or equal to 5000 mg, etc. In some embodiments, the composition may be applied at a dosage of less than or equal to 5000 mg, less than or equal to 3000 mg, less than or equal to 2500 mg, less than or equal to 2000 mg, less than or equal to 1500 mg, less than or equal to 1000 mg, less than or equal to 500 mg, less than or equal to 300 mg, less than or equal to 200 mg, less than or equal to 100 mg, less than or equal to 90 mg, less than or equal to 80 mg, less than or equal to 70 mg, less than or equal to 60 mg, less than or equal to 50 mg, less than or equal to 40 mg, less than or equal to 30 mg, less than or equal to 25 mg, less than or equal to 20 mg, less than or equal to 15 mg, less than or equal to 10 mg, etc. Combinations of these ranges are also possible. For example, the dosage may be greater than or equal to 10 mg and less than or equal to 20 mg, greater than or equal to 50 mg and less than or equal to 100 mg, or greater than or equal to 100 mg and less than or equal to 200 mg.

In some embodiments, the compositions may be given to a subject (e.g., a human) at a dosage of greater than or equal to 1 mg/kg, greater than or equal to 2 mg/kg, greater than or equal to 3 mg/kg, greater than or equal to 4 mg/kg, greater than or equal to 5 mg/kg, greater than or equal to 6 mg/kg, greater than or equal to 7 mg/kg, greater than or equal to 8 mg/kg, greater than or equal to 9 mg/kg, greater than or equal to 10 mg/kg, greater than or equal to 11 mg/kg, greater than or equal to 12 mg/kg, greater than or equal to 13 mg/kg, greater than or equal to 14 mg/kg, or greater than or equal to 15 mg/kg. In some embodiments, the statins maybe given at a dosage of less than or equal to 15 mg/kg, less than or equal to 14 mg/kg, less than or equal to 13 mg/kg, less than or equal to 12 mg/kg, less than or equal to 11 mg/kg, less than or equal to 10 mg/kg, less than or equal to 9 mg/kg, less than or equal to 8 mg/kg, less than or equal to 7 mg/kg, less than or equal to 6 mg/kg, less than or equal to 5 mg/kg, less than or equal to 4 mg/kg, less than or equal to 3 mg/kg, less than or equal to 2 mg/kg, or less than or equal to 1 mg/kg. Combinations of these ranges are also possible. For example, in some embodiments, the dosage is greater than or equal to 1 mg/kg and less than or equal to 15 mg/kg, greater than or equal to 1 mg/kg and less than or equal to 10 mg/kg, greater than or equal to 5 mg/kg or less than or equal to 10 mg/kg.

In another aspect, a composition as described herein may be administered to a subject. The composition may be administered by itself. In some embodiments, the composition is administered in conjunction with co-factors, other therapeutics, or the like.

The compositions may be applied in a therapeutically effective, pharmaceutically acceptable amount as a pharmaceutically acceptable formulation, for example, a pharmaceutically acceptable carrier such as those described below. The term “effective amount” of a composition, such as the compositions described herein, refers to the amount necessary or sufficient to realize a desired biologic effect. For example, an effective amount of a composition to treat a tumor may be an amount sufficient to reduce the tumor's size. Combined with the teachings provided herein, by choosing among the various active compositions and weighing factors such as potency, relative bioavailability, patient body weight, severity of adverse side effects and mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular compositions being administered the size of the subject, or the severity of the diseaseor condition. One of ordinary skill in the art can empirically determine the effective amount of the compositions without necessitating undue experimentation.

The terms “treat,” “treated,” “treating,” and the like, when used herein, refer to administration of the compositions to a subject which may increase the resistance of the subject to a disease, or to further progression of the disease, to control progression of the disease, and/or slow the progression of or to reduce the severity of symptoms of the disease. The effective amount may depend on the particular disease being treated and the desired outcome.

In some embodiments, a therapeutically effective dose of a composition can be initially determined from an animal model. The applied dose may be adjusted based on the relative bioavailability and potency of the administered compound. Adjusting the dose to achieve maximal efficacy based on the methods described above and other methods as are well-known in the art is well within the capabilities of the ordinarily skilled artisan.

In administering the composition to a subject, dosing amounts, dosing schedules, routes of administration, and the like may be selected so as to affect known activities of these compositions. Dosages may be estimated based on the results of experimental models, optionally in combination with the results of assays of compositions described herein. Dosage may be adjusted appropriately to achieve desired drug levels, local or systemic, depending upon the mode of administration. The doses may be given in one or several administrations per day. Multiple doses per day may be used to achieve appropriate systemic levels of the compositions within the subject or within the cell or virus targeted by the composition.

The dose may be chosen to provide a therapeutically effective amount of the composition to the cell or virus targeted by the composition. The dosage may be given in a maximum safe dose. The maximum safe dose may be chosen such that a high therapeutically effective amount of the composition is delivered to the subject but such that a risk to the subject of potentially detrimental side effects is minimal.

The dose of the composition may be chosen to have a desired concentration at the targeted virus or cell, to have a desired efficacy, to have a desired longevity within the subject, to have a desired rate of administration, to have a desired frequency of administration, to act in an appropriate fashion when administered concurrently with other treatments (e.g., as in a cocktail), or for any of a variety of other purposes known to those of ordinary skill in the art.

The dose may be chosen based, at least in part, on conditions associated with the subject. For example, the dose may be chosen based at least in part on the species, age, sex, weight, size, environment, metabolism, physical condition, or current state of health of the subject. In some cases, a subject-specific maximum dose may be used. In some cases, the dose may be administered in a way that limits deleterious effects to the subject (e.g., the dose may be delivered orally, nasally, intravenously, etc., depending on the needs of the subject).

Doses of the composition may be administered daily, weekly, or monthly and any other amount of time therebetween.

A dose may include the composition in any appropriate amount. In some embodiments, the dose of the composition is greater than or equal to 0.1 micrograms, greater than or equal to 0.5 micrograms, greater than or equal to 10 micrograms, greater than or equal to 50 micrograms, greater than or equal to 100 micrograms, greater than or equal to 500 micrograms, greater than or equal to 1000 micrograms, or more. In some embodiments, the dose of the composition is less than or equal to 10000 micrograms, less than or equal to 5000 micrograms, less than or equal to 1000 micrograms, less than or equal to 500 micrograms, less than or equal to 100 micrograms, less than or equal to 50 micrograms, or less. Combinations of these ranges are also possible. For example, in some embodiments the dose of the composition is greater than or equal to 0.5 micrograms and less than or equal to 10000 micrograms.

Doses of the composition may be administered with any of a variety of average rates. In some embodiments, the doses are administered at a rate of greater than or equal to 0.01 micrograms/hour, greater than or equal to 0.05 micrograms/hour, greater than or equal to 0.1 micrograms/hour, greater than or equal to 0.5 micrograms/hour, greater than or equal to 1 micrograms/hour, greater than or equal to 5 micrograms/hour, greater than or equal to 10 micrograms/hour, greater than or equal to 50 micrograms/hour, greater than or equal to 100 micrograms/hour, greater than or equal to 500 micrograms/hour, greater than or equal to 1000 micrograms/hour, or greater. In some embodiments, the doses are administered at a rate of less than or equal to 10000 micrograms/hour, less than or equal to 5000 micrograms/hour, less than or equal to 1000 micrograms/hour, less than or equal to 500 micrograms/hour, less than or equal to 100 micrograms/hour, less than or equal to 50 micrograms/hour, less than or equal to 10 micrograms/hour, less than or equal to 1 micrograms/hour, less than or equal to 0.5 micrograms/hour, less than or equal to 0.1 micrograms/hour, or less. Combinations of these ranges are possible. For example, in some embodiments, the doses are administered at a rate of greater than or equal to 0.01 micrograms/hour, and less than or equal to 10000 micrograms/hour.

The composition may be administered over extended period of time. In some embodiments, the dose is administered over a period of greater than or equal to 1 h, greater than or equal to 2 h, greater than or equal to 4 h, greater than or equal to 6 h, greater than or equal to 12 h, greater than or equal to 24 h, greater than or equal to 3 days, greater than or equal to 7 days, greater than or equal to 10 days, greater than or equal to 14 days, greater than or equal to 21 days, greater than or equal to 30 days, greater than or equal to 45 days, greater than or equal to 60 days, greater than or equal to 90 days, or greater. In some embodiments, the dose is administered over a period of less than or equal to 365 days, less than or equal to 200 days, less than or equal to 90 days, less than or equal to 60 days, less than or equal to 45 days, less than or equal to 30 days, less than or equal to 21 days, less than or equal to 14 days, less than or equal to 10 days, less than or equal to 7 days, less than or equal to 3 days, less than or equal to 24 h, less than or equal to 12 h, or less. Combinations of these ranges are possible. For example, in some embodiments, the dose is administered over a period of greater than or equal to 1 h and less than or equal to 365 days.

The subject may be any appropriate subject. For example the subject may be a mammal, such as a human, or a non-human animal, such as a dog, a cat, a horse, a rabbit, a cow, a pig, a sheep, a goat, a rat (e.g., Rattus Norvegicus), a mouse (e.g., Mus musculus), a guineapig, a non-human primate (e.g., a monkey, a chimpanzee, a baboon, an ape, a gorilla, etc.), or the like.

Administration of a composition of the disclosure may be accomplished by any of a variety of medically acceptable methods that allows the composition to reach its target cell or virus. The particular mode selected may depend of course, upon factors such as those previously described, for example, the particular composition, the severity of the state of the subject being treated, the dosage required for therapeutic efficacy, etc. As used herein, a “medically acceptable” mode of treatment is a mode able to produce effective levels of the compositions within the subject without causing clinically unacceptable adverse effects. In the case of neurodegenerative diseases the blood brain barrier may prevent the agent from reaching the senescent cells. In this case themedically acceptable mode may be administration through the cerebral spinal fluid, i.e., intrathecally.

Any medically of a variety of acceptable methods may be used to administer the compositions to the subject. The administration may be localized (e.g., to a particular region, physiological system, tissue, organ, or cell type) or systemic, depending on the condition to be treated. For example, the compositions may be administered orally, vaginally, rectally, buccally, pulmonary, topically, nasally, transdermally, through parenteral injection or implantation, via surgical administration, or any other method of administration where access to the tumor is achieved. In some cases, more than one method of administration may be used, e.g., if two or more compositions are to be administered.

Examples of parenteral methods of administration that can be used include intravenous, intradermal, subcutaneous, intracavity, intramuscular, intraperitoneal, epidural, or intrathecal administration. Examples of implantation methods of administration include implantation or injection of any implantable or injectable drug delivery system. Oral administration may be used in some embodiments. Oral administration may be advantageous because of the convenience to the subject as well as the dosing schedule. A compositions suitable for oral administration may be administered using discrete units, such as hard or soft capsules, pills, cachettes, tablets, troches, dissolving films or lozenges. Other oral compositions suitable for use include solutions or suspensions such as a syrup, an elixir, or an emulsion. The solutions or suspensions may comprise aqueous or non-aqueous liquids. In some embodiments, a composition may be used to fortify a food or a beverage.

The compositions, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions, or emulsions in oily or aqueous vehicles, and may contain formulatory agents such as suspending, stabilizing and/or dispersing agents.

Pharmaceutical formulations for parenteral administration may include aqueous solutions of the active compounds in water soluble form. Suspensions of the active compounds may be prepared, in some cases, as appropriate oily injection suspensions. Suitable lipophilic solvents or vehicles may include, but are not limited to, fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. The suspension may contain suitable stabilizers. In some embodiments, the suspension comprises agents that increase the solubility of the compounds. The stabilizers or solubility increasers may allow for the preparation of highly concentrated solutions.

The composition may be formulated as a depot preparation in some embodiments. Such long-acting formulations may be formulated, in some cases, with suitable polymeric or hydrophobic materials (for example, as an emulsion in an acceptable oil) or ion exchange resins, oras sparingly soluble derivatives, for example, as a sparingly soluble salt.

The composition may be prepared in the form of a liquid pharmaceutical preparation. In some embodiments, the liquid pharmaceutical preparation comprises a solution, a suspension, or an emulsion. The composition may be included in the liquid pharmaceutical preparation in any of a variety of forms. For example, the composition may be comprised by an aqueousor saline solution (e.g., suitable for inhalation), may be microencapsulated, may be encochleated, may be coated onto microscopic gold particles, may be contained in liposomes, may be nebulized, may be aerosolized, or may be dried onto a sharp object to be scratched into the skin.

The composition may be prepared in the form of a solid pharmaceutical preparation. For example, the composition may be included within granules; powders; tablets; coated tablets; (micro) capsules; suppositories; syrups; emulsions; suspensions; creams; or dropsor preparations with protracted release of active compounds. In some embodiments, the composition is prepared in the form of a solid pharmaceutical preparation configured to be dissolved or otherwise converted to a pharmaceutical liquid preparation. For example, the composition may be prepared in the form of a dry powder that may be dissolved in water to form an aqueous solution. Liquid or solid compositions may include an additive such as an excipient. In some embodiments, the additive comprises disintegrants, binders, coatingagents, swelling agents, lubricants, flavorings, sweeteners or solubilizers, although any of a variety of additives may be used and the disclosure is not so limited.

In some embodiments, a composition may be administered to provide sequential exposures to a composition over a certain time period, for example, hours, days, weeks, months or years.

For example, the composition may be administered by repeated administrations or by controlled release. For example, the composition may be control-released over the time-period such that it sustains a dosage of the composition during that time-period. Control release may be achieved, for example, by oral dosage forms, bolus injections, transdermal patches, subcutaneous implants, or other methods such as those described herein. Maintaining a substantially constant concentration of a composition may be desired insome cases.

Other delivery systems suitable for use in certain embodiments may include time-release, delayed release, sustained release, or controlled release delivery systems. Such systems may advantageously avoid the need for repeated administrations, increasing convenience to the subject. Many types of delivery systems are available and known to those of ordinary skill in the art. The composition may be administered, for example, using a delivery system such as a polymer-based systems such as polylactic and/or polyglycolic acids, polyanhydrides, polycaprolactones and/or combinations of these; nonpolymer systems that are lipid-based including sterols such as cholesterol, cholesterol esters, and fatty acids or neutral fats such as mono-, di- and triglycerides; hydrogel release systems; liposome-based systems; phospholipid based-systems; silastic systems; peptide based systems; wax coatings; compressed tablets using conventional binders and excipients; or partially fused implants. Specific examples include, but are not limited to, erosional systems in which the composition is contained in a form within a matrix, or diffusional systems in which an active component controls the release rate. The formulation may be present as, for example, microspheres, hydrogels, polymeric reservoirs, cholesterol matrices, or polymeric systems, etc. In some embodiments, the system may allow sustained or controlled release of a composition to occur, for example, through control of the diffusion or erosion/degradation rate of the formulation. In addition, a pump-based hardware delivery system may be used in some embodiments.

In some embodiments, the composition is delivered using an implant. The implant may be configured for short-term or long-term release of the composition. In some embodiments, the delivery system is a long-term release implant. Use of a long-term release implant may be particularly suitable in some embodiments. “Long-term release,” as used herein, means that an implant containing a composition as described herein is constructed and arranged to deliver therapeutically effective levels for In some embodiments, the composition is delivered at therapeutically effective levels over a period of greater than or equal to 1 h, greater than or equal to 2 h, greater than or equal to 4 h, greater than or equal to 6 h, greater than or equal to 12 h, greater than or equal to 24 h, greater than or equal to 3 days, greater than or equal to 7 days, greater than or equal to 10 days, greater than or equal to 14 days, greater than or equal to 21 days, greater than or equal to 30 days, greater than or equal to 45 days, greater than or equal to 60 days, greater than or equal to 90 days, or greater. In some embodiments, the composition is delivered at therapeutically effective levels over a period of less than or equal to 365 days, less than or equal to 200 days, less than or equal to 90 days, less than or equal to 60 days, less than or equal to 45 days, less than or equal to 30 days, less than or equal to 21 days, less than or equal to 14 days, less than or equal to 10 days, less than or equal to 7 days, less than or equal to 3 days, less than or equal to 24 h, less than or equal to 12 h, or less. Combinations of these ranges are possible. For example, in some embodiments, In some embodiments, the composition is delivered at therapeutically effective levels over a period of greater than or equal to 1 h and less than or equal to 365 days. Long-term release implants are well known to those of ordinary skill in the art, and include some of the release systems described herein.

In certain embodiments a composition can be combined with a suitable pharmaceutically acceptable carrier. In general, pharmaceutically acceptable carriers suitable for use are well-known to those of ordinary skill in the art. As used herein, a “pharmaceutically acceptable carrier” refers to a non-toxic material that does not significantly interfere with the effectiveness of the biological activity of the active compound(s) to be administered, but is used as a formulation ingredient, for example, to stabilize or protect the active compound(s) within a composition before use. For example the composition may be incorporated into a liposome, incorporated into a polymer release system, suspended in a liquid (e.g., in a dissolved form or a colloidal form), or other methods such as those described herein. The carrier may include one or more compatible solid or liquid fillers, diluents or encapsulating substances which are suitable for administration to a human or other vertebrate animal. The term “carrier” denotes an organic or inorganic ingredient, which may be natural or synthetic, with which one or more active compounds of the disclosure are combined to facilitate application. The carrier may beco-mingled or otherwise mixed with one or more compositions as described herein, and/or with each other, in a manner such that there is no interaction which would substantially impair the desired pharmaceutical efficacy. The carrier may be either soluble or insoluble, depending on the application. Examples of well-known carriers include, but are not limited to, glass, polystyrene, polypropylene, polyethylene, dextran, nylon, amylase, natural and modified cellulose, polyacrylamide, agarose and magnetite. The nature of the carrier may be either soluble or insoluble.

The formulations described herein may be administered in pharmaceutically acceptable solutions, in some embodiments. Pharmaceutically acceptable solutions may routinely contain pharmaceutically acceptable concentrations of salt, buffering agents, preservatives, compatible carriers, adjuvants, emulsifiers, diluents, excipients, chelating agents, fillers, drying agents, antioxidants, antimicrobials, preservatives, binding agents, bulking agents, silicas, solubilizers, stabilizers and optionally other therapeutic ingredients, that may be used with the composition. For example, if the formulation is a liquid, the carrier may be a solvent, partial solvent, or non-solvent, and may be aqueous or organically based. Non-limiting examples of suitable formulation ingredients include diluents such as calcium carbonate, sodium carbonate, lactose, kaolin, calcium phosphate, or sodium phosphate; granulating and disintegrating agents such as cornstarch or algenic acid; binding agents such as starch, gelatin or acacia; lubricating agents such as magnesium stearate, stearic acid, or talc; time-delay materials such as glycerol monostearate or glycerol distearate; suspending agents such as sodium carboxymethylcellulose, methylcellulose, hydroxypropylmethylcellulose, sodium alginate, polyvinylpyrrolidone; dispersing or wetting agents such as lecithin or other naturally-occurring phosphatides; thickening agents such as cetyl alcohol or beeswax; buffering agents such as acetic acid and salts thereof, citric acid and salts thereof, boric acid and salts thereof, or phosphoric acid and salts thereof; or preservatives such as benzalkonium chloride, chlorobutanol, parabens, or thimerosal. The compositions of the disclosure may be formulated into preparations in solid, semi-solid, liquid or gaseous forms such as tablets, capsules, elixirs, powders, granules, ointments, solutions, depositories, inhalants or injectables, etc.

Suitable buffering agents include, but are not limited to: acetic acid and a salt (1-2% w/v); citric acid and a salt (1-3% w/v); boric acid and a salt (0.5-2.5% w/v); and phosphoric acid and a salt (0.8-2% w/v). Suitable preservatives include, but are not limited to, benzalkonium chloride (0.003-0.03% w/v); chlorobutanol (0.3-0.9% w/v); parabens (0.01-0.25% w/v) and thimerosal (0.004-0.02% w/v).

Preparations may include solutions, suspensions, or emulsions as described above. The solutions, suspensions, or emulsions may be sterile. In some embodiments, the solutions, suspensions, or emulsions are aqueous. In some embodiments the solutions, suspensions or emulsions are non-aqueous. The sterile aqueous or nonaqueous solutions, suspensions, or emulsions may be isotonic with the blood of the subject in certain embodiments. Non-limiting examples of nonaqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or di-glycerides. The solution, suspension, or emulsion may include an aqueous carrier. Exemplary aqueous carriers that may be used include, but are not limited to, water, alcoholic/aqueous solutions, emulsions or suspensions, including saline and buffered media. The preparation may further comprise a parenteral vehicle configured to improve suitability of a solution, suspension, or emulsion for parenteral delivery. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present in some embodiments, such as, for example, antimicrobials, antioxidants, chelating agents and inert gases and the like.

In some embodiments, a composition as described herein may be brought into association or contact with a suitable carrier. The suitable carrier may comprise one or more accessory ingredients. The preparation may be prepared by any of a variety of suitable techniques. For example, the preparation may be prepared by uniformly and intimately associating a composition with a liquid carrier, a finely divided solid carrier or both. In some embodiments, the resulting preparation may be shaped (e.g., into a tablet, a pellet, etc.).

The compositions as discussed herein may be administered per se (neat) or in the form of a pharmaceutically acceptable salt. Although non-pharmaceutically acceptable salts may be used for preparing compositions, when used in medicine salts should be pharmaceutically acceptable. The term “pharmaceutically acceptable salts” includes salts of compositions described herein, prepared in combination with, for example, acids or bases. Pharmaceutically acceptable salts can be prepared as alkaline metal salts, such as lithium, sodium, or potassium salts; or as alkaline earth salts, such as magnesium or calcium salts. Examples of suitable bases that may be used to form salts include ammonium, or mineral bases such as sodium hydroxide, lithium hydroxide, potassium hydroxide, calcium hydroxide, magnesium hydroxide, and the like. Examples of suitable acids that may be used to form salts include inorganic or mineral acids such as hydrochloric, hydrobromic, hydroiodic, hydrofluoric, nitric, carbonic, monohydrogencarbonic, phosphoric, monohydrogenphosphoric, dihydrogenphosphoric, sulfuric, monohydrogensulfuric, phosphorous acids and the like. Other suitable acids include organic acids, for example, acetic, propionic, isobutyric, maleic, malonic, benzoic, succinic, suberic, fumaric, mandelic, phthalic, benzenesulfonic, p-tolylsulfonic, citric, tartaric, methanesulfonic, glucuronic, galacturonic, salicylic, formic, naphthalene-2-sulfonic, and the like. Still other suitable acids include amino acids such as arginate, aspartate, glutamate, and the like. Also, such salts can be prepared as alkaline metal or alkaline earth salts, such as sodium, potassium or calcium salts of the carboxylic acid group.

In another aspect, the present disclosure also provides any of the above-mentioned compositions in kits, optionally including instructions for use of the composition for the treatment of a disease or condition (e.g., by targeting a senescent cell, a fat cell, plaque, or a virus such as HIV or SARS-COV-2). In some cases, the kit can include a description of use of the compositions as discussed herein. The kit also can include instructions for use of a combination of two or more compositions. Instructions also may be provided for administering the compositions by any suitable technique as previously described, for example, orally, intravenously, pump or implantable delivery device, or via another known route of drug delivery.

The kits described herein may also contain one or more containers. The container may contain compositions and other ingredients as previously described. The kits also may contain instructions for mixing, diluting, and/or administrating the compositions of the disclosure in some cases. The kits also can include other containers with one or more solvents, surfactants, preservative and/or diluents (e.g., normal saline (0.9% NaCl), or 5% dextrose) as well as containers for mixing, diluting or administering the components in a sample or to a subject in need of such treatment.

The compositions of the kit may be provided as any suitable form, for example, as liquid pharmaceutical preparations or as solid pharmaceutical preparations. When the composition provided is a dry powder, the composition may be reconstituted by the addition of a suitable solvent, which may also be provided in some cases. In embodiments where liquid forms of the composition are used, the liquid form may be concentrated or ready to use. The solvent will depend on the composition and the mode of use or administration. Suitable solvents for drug compositions are well known, for example as previously described, and are available in the literature. The solvent will depend on the composition and the mode of use or administration.

In still another aspect, the disclosure includes the promotion of one or more of the above-described embodiments, e.g., in vitro or in vivo, promotion of treatment or prevention of a tumor, e.g., by administering, to a subject, compositions such as those described herein.

The following examples are intended to illustrate certain embodiments of the present disclosure, but do not exemplify the full scope of the disclosure.

An antibody, as described herein, may comprise a biopolymer, such as a polypeptide. For example, the antibody may comprise a protein. In some embodiments, the antibody is a glycoprotein. However, in some embodiments, the antibody comprising a protein is a glycoprotein. The antibody may be substantially encoded by immunoglobulin genes or fragments of immunoglobulin genes. Any of a variety of immunoglobulin genes or fragments thereof are known to those of ordinary skill in the art. For example, the antibody may be substantially encoded by immunoglobulin genes include the kappa, lambda, alpha, gamma, delta, epsilon, and mu constant region genes, as well as myriad immunoglobulin variable region genes and fragments thereof. The antibody may be encoded by a light chain immunoglobulin gene or a fragment thereof. Light chain immunoglobulins may be classified as either kappa or lambda.

The antibody may be encoded by a heavy chain immunoglobulin gene or a fragment thereof. Heavy chain immunoglobulins may be classified as gamma, mu, alpha, delta, or epsilon (which in turn define the immunoglobulin classes, IgG, IgM, IgA, IgD and IgE, respectively).

An immunoglobulin structural unit may comprise a tetramer. Each tetramer may be composed of two identical pairs of polypeptide chains, each pair having one light chain and one heavy chain.

In some embodiments, the light chain has a molecular weight of greater than or equal to 15 kDa, greater than or equal to 20 kDa, greater than or equal to 25 kDa, or greater. In some embodiments, the light chain has a molecular weight of less than or equal to 35 kDa, less than or equal to 30 kDa, less than or equal to 25 kDa, or less. Combinations of these ranges are possible. For example, in some embodiments, the light chain has a molecular weight of greater than or equal to 15 kDa and less than or equal to 35 kDa.

In some embodiments, the heavy chain has a molecular weight of greater than or equal to 50 kDa, greater than or equal to 55 kDa, greater than or equal to 60 kDa, or greater. In some embodiments, the heavy chain has a molecular weight of less than or equal to 70 kDa, less than or equal to 65 kDa, less than or equal to 60 kDa, or less. Combinations of these ranges are possible. For example, in some embodiments, the heavy chain has a molecular weight of greater than or equal to 50 kDa and less than or equal to 70 kDa.

The N-terminus of each polypeptide chain may define a variable region of the immunoglobulin structural unit. The variable region may be primarily responsible for antigen recognition. In some embodiments, the variable region comprises greater than or equal to 95, greater than or equal to 98, greater than or equal to 100, greater than or equal to 103, greater than or equal to 105, or more amino acids. In some embodiments, the variable region comprises less than or equal to 115, less than or equal to 113, less than or equal to 110, less than or equal to 108, less than or equal to 105, or fewer amino acids. Combinations of these ranges are possible. For example, in some embodiments, the variable region comprises of greater than or equal to 95 and less than or equal to 115 amino acids.

Antibodies may exist as intact immunoglobulins. However, in some embodiments, antibodies exist as any of a number of immunoglobulin fragments.

The immunoglobulin fragment may be produced by digestion with any of a variety of peptidases (e.g., pepsin). For example, in some embodiments immunoglobulin fragments may be formed by digesting an antibody using pepsin. In some, exemplary embodiments, pepsin is used to digest the Fc domain of an antibody, e.g., by degrading disulfide linkages in a hinge region of the Fc domain to produce F(ab)′2. The F(ab)′2 is, according to certain embodiments, a dimer of Fab which itself is a light chain joined to VH-CH1 by a disulfide bond. The F(ab)′2 may be reduced to break the disulfide linkage in the hinge region, thereby converting the (Fab′) 2 dimer into a Fab′ monomer. The Fab′ monomer, according to some embodiments, comprises Fab and a part of the hinge region of the Fc domain.

In some embodiments, the immunoglobulin fragment is synthesized de novo. The immunoglobulin fragment may be produced by any of a variety of methods known to those of ordinary skill in the art, such as by chemical synthesis, by utilizing recombinant DNA methodology, or by “phage display” methods.

Examples of antibodies include single chain antibodies, e.g., single chain Fv (scFv) antibodies in which a variable heavy and a variable light chain are joined together (directly or through a peptide linker) to form a continuous polypeptide. In one embodiment, the antibody is a monoclonal antibody.

In some embodiments, the composition comprising the antibody or antibody fragment is configured such that the antibody or antibody fragment retains a relatively high affinity for a target molecule. Without wishing to be bound by theory, the affinity of the antibody or antibody fragment for the target may be inversely related to the dissociation constant (K_{D}) between the antibody or antibody fragment and the target, so that a lower K_{D} value corresponds to a higher affinity. In some embodiments, the K_{D} value of the antibody or antibody fragment is less than or equal to 10⁻⁶ M, less than or equal to 10⁻⁷ M, less than or equal to 10⁻⁸ M, less than or equal to 10⁻⁹ M, less than or equal to 10⁻¹⁰ M, or less under physiological conditions. In some embodiments, the K_{D} value of the antibody or antibody fragment is greater than or equal to 10⁻¹³ M, greater than or equal to 10⁻¹² M, greater than or equal to 10⁻¹¹ M, or more. Combinations of these ranges are also possible. For example, in some embodiments, the K_{D} value of the antibody or the antibody fragment is greater than or equal to 10⁻¹² M and less than or equal to 10⁻⁶ M. The K_{D} value of the composition may be determined by a test that would be well-known to one of ordinary skill in the art.

In some embodiments, the K_{D} of the composition may be relatively close to the K_{D} of the pure antibody or antibody fragment analogous to the antibody or antibody fragment of the composition. This relatively high affinity of the composition for its target may be associated with the fact that the composition is a fusion protein.

In this context, the term “antibody fragment” can refer to at least one portion of an antibody, that retains the ability to specifically interact with (e.g., by binding, steric hindrance, stabilizing/destabilizing, spatial distribution) an epitope of an antigen. Examples of antibody fragments include, but are not limited to, any antibody fragments described elsewhere herein and including Fab, Fab′, F(ab′)₂, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv.

In some embodiments, the antibody may be a minibody or a nanobody. In the context of the present disclosure, it has been inventively recognized that use of a minibody or a nanobody may advantageously increase the binding affinity of the antibody, relative to a full-sized antibody.

The following examples are intended to illustrate certain embodiments of the present invention, but do not exemplify the full scope of the invention.

### Example 1

In one prophetic example, a 60 year old male with decreased stamina (e.g., as a symptom of aging) is injected with a composition comprising a fusion protein containing calreticulin and an antibody to senescent muscle cells. The fusion protein binds to senescent cells and the senescent cell/fusion protein composition is recognized by antigen presenting cells such as dendritic cells, incorporated into such cells, and carried to the lymph nodes where the virus educates T cells to recognize it. Such T cells are then effective in seeking out and killing other senescent cells. A subject so treated has his ability to walk longer distances restored.

### Example 2

In one prophetic example, a 60 year old male with 90% narrowing of the left anterior descending artery (LAD) is injected with a composition comprising a fusion protein comprising calreticulin and an antibody to plaque. The fusion protein binds to plaque and the plaque/fusion protein composition is recognized by antigen presenting cells such as dendritic cells, incorporated into such cells and is carried to the lymph nodes where the virus educates T cells to recognize it. Such T cells are then effective in seeking out and removing plaque. A subject so treated could experience reduced LAD stenosis from 90% to 45% in 60 days.

### Example 3

In one prophetic example, a 60 year old male with a body mass index of 38 is injected with a composition comprising a fusion protein comprising calreticulin and an antibody to fat cells. The fusion protein binds to fat cells and the fat cell/fusion protein composition is recognized by antigen presenting cells such as dendritic cells, incorporated into such cells and is carried to the lymph nodes where the virus educates T cells to recognize it. Such T cells are then effective in seeking out and killing other fat cells. A subject so treated could experience reduced body mass index to 34 in two months.

### Example 4

In one prophetic example, a person infected with SARS-COV-2 is injected with a composition comprising a fusion protein containing calreticulin and an antibody to SARS-COV-2. The fusion protein binds to virus and the virus/fusion protein composition is recognized by antigen presenting cells such as dendritic cells, incorporated into such cells and is carried to the lymph nodes where the virus educates T cells to recognize it. Such T cells are then effective in seeking out and killing virus. A subject so treated has disease treated and becomes immunized against future exposure to the same virus.

### Example 5

In this prophetic example, a composition comprising a fusion protein comprising an eat-me signal such as calreticulin and a surface antigen of senescent cells is injected into a subject. Antigen presenting cells such as dendritic cells recognize the composition and the composition is incorporated into the dendritic cells. The dendritic cells carry the composition to the lymph nodes where it educates T cells to recognize the senescent cells. The educated T cells then kill the senescent cells whenever they are encountered. Such a subject is immunized against aging.

### Example 6

In this prophetic example, a composition comprising a fusion protein comprising an eat-me signal such as calreticulin and a surface antigen of plaque is injected into a subject. Antigen presenting cells such as dendritic cells recognize the composition and the composition is incorporated into the dendritic cells. The dendritic cells carry the composition to the lymph nodes where it educates T cells to recognize the plaque. The educated T cells then removes the plaque whenever they are encountered. Such a subject is immunized against atherosclerosis.

### Example 7

In this prophetic example, a composition comprising a fusion protein comprising an eat-me signal such as calreticulin and a surface antigen of fat cells is injected into a subject. Antigen presenting cells such as dendritic cells recognize the composition and the composition is incorporated into the dendritic cells. The dendritic cells carry the composition to the lymph nodes where it educates T cells to recognize the fat cells. The educated T cells then kill the fat cells whenever they are encountered. Such a subject is immunized against obesity.

### Example 8

In this prophetic example, a composition comprising a fusion protein comprising an eat-me signal such as calreticulin and a fragment of a virus such as SARS-COV-2 is injected into a subject. Antigen presenting cells such as dendritic cells recognize the composition and the composition is incorporated into the dendritic cells. The dendritic cells carry the composition to the lymph nodes where it educates T cells to recognize the virus. The educated T cells then kill the virus whenever it is encountered. Such a subject is immunized against the particular virus such as SARS-COV-2.

### Example 9

In this prophetic example, a composition comprising a fusion protein comprising an eat-me signal such as calreticulin and mRNA, where the mRNA is mRNA that expresses a surface antigen of senescent cells. The fusion protein is taken up by dendritic cells through the receptor LPR of the dendritic cells.

### Example 10

In this prophetic example, a composition comprising a fusion protein comprising an eat-me signal such as calreticulin and mRNA, where the mRNA is mRNA that expresses a surface antigen of plaque. The fusion protein is taken up by dendritic cells through the receptor LPR of the dendritic cells.

### Example 11

In this prophetic example, a composition comprising a fusion protein comprising an eat-me signal such as calreticulin and mRNA, where the mRNA is mRNA that expresses a surface antigen of fat cells. The fusion protein is taken up by dendritic cells through the receptor LPR of the dendritic cells.

### Example 12

In this prophetic example, a composition comprising a fusion protein between an eat-me signal such as calreticulin and mRNA where the mRNA is mRNA that expresses a spike protein of SARS-COV-2. The fusion protein is taken up by dendritic cells through the receptor LPR of the dendritic cells.

### Example 13

In this prophetic example, a composition comprising a fusion protein comprising calreticulin and one or more fragments of surface antigens of senescent cells is dissolved in a deep eutectic solvent such as a 1:2 composition of choline chloride and propylene glycol. The composition comprising the deep eutectic solvent and the fusion protein is incorporated into a thin dissolving film and applied to an inside of a cheek of a subject. The fusion protein vaccine transmitted into the subject's blood transbuccally.

### Example 14

In this prophetic example, a composition comprising a fusion protein comprising calreticulin and one or more fragments of surface antigens of plaque is dissolved in a deep eutectic solvent such as a 1:2 composition of choline chloride and propylene glycol. The composition comprising the deep eutectic solvent and the fusion protein is incorporated into a thin dissolving film and applied to an inside of a cheek of a subject. The fusion protein vaccine transmitted into the subject's blood transbuccally.

### Example 15

In this prophetic example, a composition comprising a fusion protein comprising calreticulin and one or more fragments of surface antigens of fat cells is dissolved in a deep eutectic solvent such as a 1:2 composition of choline chloride and propylene glycol. The composition comprising the deep eutectic solvent and the fusion protein is incorporated into a thin dissolving film and applied to an inside of a cheek of a subject. The fusion protein vaccine transmitted into the subject's blood transbuccally.

### Example 16

In this prophetic example, a composition comprising a fusion protein comprising calreticulin and one or more fragments of the spike protein of SARS-COV-2 are dissolved in a deep eutectic solvent such as a 1:2 composition of choline chloride and propylene glycol. The composition comprising the deep eutectic solvent and the fusion protein is incorporated into a thin dissolving film and applied to an inside of a cheek of a subject. The fusion protein vaccine transmitted into the subject's blood transbuccally.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present disclosure.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, “wt %” is an abbreviation of weight percentage. As used herein, “at %” is an abbreviation of atomic percentage.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A composition for treating or preventing a disease or condition, the composition comprising:
a fusion protein comprising an eat-me signal and at least a portion of an antibody,
wherein the at least a portion of the antibody is configured to bind to a cell, a virus, or an extracellular material associated with a disease or condition.

2. The composition of claim 1, wherein the eat-me signal triggers an immune response against the cell, the virus, or the extracellular material.

3. A method of treating or preventing a disease or condition, comprising:
administering to a subject a composition comprising a fusion protein that comprises an eat-me signal and an at least a portion of an antibody.

4. The method of claim 3, wherein the at least a portion of the antibody recognizes a cell, a virus, or an extracellular material associated with a disease or condition of the subject, and triggers an immune response against the cell, the virus, or the extracellular material.

5. A composition for treating or preventing a disease or condition, the composition comprising:
a fusion protein comprising an eat-me signal and a fragment of a surface antigen of a cell or virus,
wherein the composition is configured to trigger an immune response against the cell or the virus.

6. A method of treating or preventing a disease or condition, comprising:
administering to a subject a composition comprising a fusion protein that comprises an eat-me signal and a fragment of a surface antigen of a cell or virus.

7. The method of claim 6, wherein the composition triggers an immune response against the cell or the virus.

8. The composition or method of any one of claims 1-4, wherein the at least a portion of the antibody is a complete antibody.

9. The composition or method of any one of claims 1-4, wherein the at least a portion of the antibody is an antibody fragment.

10. The composition or method of any one of claims 1-4 and 8-9, wherein the at least a portion of the antibody recognizes the cell.

11. The composition or method of any one of claims 1-4 and 8-9, wherein the at least a portion of the antibody recognizes the virus.

12. The composition or method of any one of claims 1-4 and 8-9, wherein the at least a portion of the antibody recognizes the extracellular material.

13. The composition or method of any preceding claim, wherein the disease or condition is a disease, and wherein the disease is atherosclerosis or obesity.

14. The composition or method of any preceding claim, wherein the disease or condition is the condition of aging.

15. The composition or method of any one of claims 1-4 and 8-14, wherein the at least a portion of the antibody recognizes a cell, and wherein the cell is a senescent cell or a fat cell.

16. The composition or method of any one of claims 1-4 and 8-12, wherein the at least a portion of the antibody recognizes an extracellular material, and wherein the extracellular material is plaque.

17. The composition or method of any one of claims 1-4 and 8-11, the at least a portion of the antibody recognizes a virus, and wherein the virus is a coronavirus or HIV.

18. The composition or method of any one of claims 1-4 and 8-17, the at least a portion of the antibody is a minibody or a nanobody.

19. The composition or method of any one of claims 1-4 and 8-18, wherein the at least a portion of the antibody of the composition recognizes the cell, virus, or extracellular material with a K_{D} value below 10⁻⁶ M.

20. The composition or method of any one of claims 1-4 and 8-19, wherein the at least a portion of the antibody of the composition recognizes the cell, virus, or extracellular material with a K_{D} value below 10⁻⁷ M.

21. The composition or method of any one of claims 1-4 and 8-20, wherein the at least a portion of the antibody of the composition recognizes the cells, virus, or extracellular material with a K_{D} value smaller than the K_{D} value for the pure antibody.

22. A composition, comprising:
an antibody that recognizes a cell, a virus, or an extracellular material, and an eat-me signal that is recognized by antigen presenting cells such as dendric cells.

23. A composition, comprising:
an antibody fragment that recognizes particular proteins of a cell, virus, or extracellular material and an eat-me signal that is recognized by antigen presenting cells such as dendric cells.

24. The composition of any one of claims 22-23, wherein the antibody or antibody fragment recognizes the cell and the cell is a senescent cell or a fat cell, or a plaque cell.

25. The composition of any one of claims 22-24, wherein the eat-me signal is calreticulin.

26. The composition of any one of claims 1-25, wherein the antibody or the antibody fragment is directly covalently linked to the eat-me signal.

27. The composition of any one of claims 1-26, wherein a fusion protein comprises both the antibody or the antibody fragment and the eat-me signal.

28. The composition of any one of claims 1-25, wherein the antibody and the eat-me signal are linked via a linking agent.

29. A method, comprising:
administering, to a subject, a composition comprising an antibody that recognizes a cell, virus, or extracellular material and an eat-me signal.

30. A method, comprising:
administering, to a subject, a composition comprising an antibody fragment that recognizes particular proteins of a cell or virus and an eat-me signal.

31. The method of any one of claims 29-30, wherein the antibody or antibody fragment recognizes a cell and the cell is a senescent cell, a fat cell, or plaque.

32. A composition comprising:
an eat-me signal and one or more fragments of a surface antigen of a cell or virus.

33. A composition comprising:
an eat-me signal and one or more DNA fragments of a cell or virus.

34. A composition comprising:
an eat-me signal and one or more lipid fragments of a cell or virus.

35. A composition comprising:
an eat-me signal, a first component that is a DNA fragment, a lipid fragment, or a fragment of a surface antigen of a cell or virus, and a second component, different from the first component, that is also a DNA fragment, a lipid fragment, or a fragment of a surface antigen of a cell or virus.

36. The composition of any one of claims 32-35, wherein the cell is a scenescent cell, a fat cell, or plaque.

37. A composition as in any one of claims 32-36, wherein the eat-me signal is calreticulin.

38. A composition as in any one of claims 32-37 wherein the eat-me signal is a moiety recognized by antigen presenting cells.

39. A method comprising:
administering to a subject, a composition as in any one of claims 32-38.

40. A method comprising:
dissolving a composition as in any one of claims 22-38 in a deep eutectic solvent such as choline chloride/propylene glycol to produce a liquid pharmaceutical preparation and administering the liquid pharmaceutical preparation transbuccally or transdermally to a cheek of a subject.

41. The method of claim 40, wherein the liquid pharmaceutical preparation is administered using a dissolving thin film comprising the liquid pharmaceutical preparation.

42. The method of claim 40, wherein the liquid pharmaceutical preparation is part of a transdermal patch configured to be applied to the skin of a subject.
